(19) United States
(12) Patent Application Publication
(10) Pub. No.: US 2020/0181689 A1
(43) Pub. Date: Jun. 11, 2020

(54) METHOD FOR IDENTIFYING HELICOBACTER PYLORI INFECTION BY DETECTION OF THE UREC AND CAGA GENES IN STOOL SAMPLES

(71) Applicant: UNIVERSIDAD CATOLICA DEL NORTE (UCN), Antofagasta (CL)

(72) Inventors: Giuliano BERNAL DOSSETTO, Coquimbo (CL); Sergio HABERLE TAPIA, Coquimbo (CL)

(73) Assignee: Universidad Catolica Del Norte (UCN), Antofagasta (CL)

(21) Appl. No.: 16/302,811

(22) PCT Filed: May 16, 2017
(86) PCT No.: PCT/CL2017/050022

§ 371 Date: Nov. 19, 2018

(30) Foreign Application Priority Data
May 20, 2016 (CL) 1214-2016

(51) Int. Cl. C12Q 1/689

(52) U.S. Cl. CPC C12Q 1/689

(57) ABSTRACT

The invention relates to a method for identifying Helicobacter pylori infection by detection of the UreC (phosphoglucosamine mutase, SEQ ID No.9) and CagA (cytotoxin-associated gene A, sequence SEQ ID No.10) genes in stool samples. More particularly, the invention uses the aforementioned two genes as biomarkers for Helicobacter pylori infection by detecting same in stool samples in a non-invasive manner.

### FIELD OF THE INVENTION

The present invention relates to a method for the detection of specific genes of the bacterium Helicobacter pylori, in fecal samples, as non-invasive biomarkers for the identification of infection by said bacterium. The method allows to determine the presence of genetic material (DNA) of H. pylori bacteria in fecal samples of subjects, using the nested-qPCR technique, using as a marker the UreC gene (phosphoglucosamine mutase, sequence SEQ ID No: 9), and it also allows genotyping said bacterium, by means of the identification of the gene CagA (Cytotoxin associated to gene A, sequence SEQ ID No. 10), present in those more aggressive bacterial strain.

### PREVIOUS ART BACKGROUND

Currently, there are two types of tests to detect H. pylori infection, those of invasive type and those of non-invasive type. Invasive tests include culture, histology and rapid urease test (RUT). However, these tests have the disadvantage of requiring an endoscopy to take a gastric biopsy, which implies that the patient must go to a hospital to request this examination, which is not only expensive, but also involves discomfort for the patient.

The non-invasive tests include the expired air test (C13-UBT) and the analysis of fecal antigens. C13-UBT is a quick and simple method that detects the presence of H. pylori in the air exhaled by the patient, detecting the urease activity of the pathogen in the gastric mucosa. However, this technique is expensive for the patient and also requires expensive equipment to implement the technique, which is not possible in most public health services in many countries. In addition, the use of antisecretory drugs (IBO) and/or antibiotics, previous or concomitant to the test, may affect the results. The tests that detect H. pylori antigens in fecal samples present high sensitivity and specificity, and lately the costs have become more accessible to the general population. However, the accuracy of these methods decreases when the feces are watery, because H. pylori antigens are diluted. These methods are also not recommended in patients with gastric ulcer, as bleeding or micro bleeding interfere with detection.

On the other hand, neither the C13-UBT nor the detection of antigens in feces allows identifying the most aggressive strains of H. pylori. In this context, the use of qPC (quantitative PCR) for the detection of H. pylori in fecal samples is a powerful alternative to identify non-invasively the pathogen and also recognize those more aggressive strains, even in patients with gastric ulcer or samples watery fecal. The current problem is that each subject who requires a diagnosis for H. pylori infection must undergo a digestive endoscopy. This test is invasive and expensive, so many subjects avoid it. And although it is available in public health systems in many countries, these systems maintain a waiting list for this exam that can easily exceed 3 months.

On the other hand, among the specific patent documents that can be considered as sharing the same objective or a similar objective to the present invention, we can mention:

KR20030031243 presents a diagnostic method based on qPCR for the detection of H. pylori, using the primers for the genes vacA (cytotoxin vacuolizing A) and cagA (cytotoxin associated with gene A) of H. pylori. This document does not mention the type of sample for which the protocol is designed.

U.S. Pat. No. 5,928,865 describes nucleotide sequences located in the 5′ region of the CagA gene, and seeks to protect the proteins it codes for and the use of these genes and proteins for diagnosis and vaccines. US2008/076127 provides a set of oligonucleotide primers for amplifying at least one target sequence of the CagA gene of Helicobacter pylori, which are used in a method for detecting Helicobacter pylori. As a sample you can use saliva, a biopsy sample, blood, skin tissue, liquid culture, feces or urine. US2003/175746 discloses a method for the detection and/or typing of strains of Helicobacter pylori present in various biological samples, comprising the amplification of the VacA and CagA genes with suitable primers, and then the specific detection of these amplicons by hybridization with probes marked. This method is very different from ours, even though in the first part they perform PCR amplification Nor do they determine the presence of the bacteria using the UreC gene.

WO2006076010 teaches a multiplex-type polymerase chain reaction assay for detecting Helicobacter pylori in different samples. This PCR reaction is designed to simultaneously amplify a 0.86 kb DNA fragment, the Urea A gene, the 16S rRNA gene, a DNA sequence encoding a 26 kDa antigen, and the Hpa A gene. This technique differs greatly from ours in the methodological design of the PCR, also based on conventional PCR and not on qPCR.

JP2006075139 teaches a method to detect Helicobacter pylori by PCR, determining the presence of the CagA gene. However, it does not mention in which tissue it can be applied, and it is based on conventional PCR.

US2011/165576 discloses a kit and method capable of simultaneously detecting 4 Helicobacter pylori genes, one for identification. (rRNA16S Hpy), and three virulence genes (CagA, VacAml, DupA). It also discloses the starters for said kit.

Accordingly, the present invention is non-invasive, easy to implement in fecal samples (advantage over other methods), economical, is available to the entire population. On the other hand, our method allows detecting those more aggressive strains, the latter is not yet possible by known routine techniques.

### BRIEF DESCRIPTION OF THE INVENTION

The present invention relates to a method for the detection of specific genes of the bacterium Helicobacter pylori, in fecal samples, which are non-invasive biomarkers for the detection of an infection by said bacterium. The method allows to determine the presence of the genetic material (DNA) of H. pylori bacteria in fecal samples of subjects, using the nested-qPCR technique, using the UreC (Phosphoglucosamine mutase) and CagA (Cytotoxin associated to gene A) genes as markers.). In this way, it is possible to detect those more aggressive strains, associated with the development of gastric cancer (CagA positive strains [+]).

It is important to note that this technique, unlike similar ones already patented, is able to detect these genes in fecal samples, which makes it a simple and non-invasive method for the detection of H. pylori infection. On the other hand, simultaneously it allows to detect those that are CagA+strains, associated with the development of gastric cancer.

Thus, the present invention makes it possible to easily detect, and economically, in the population that requires it, the presence of H. pylori and also establish whether such infection occurs or not, by a more or less aggressive strain, which could induce the development of gastric cancer.

Another advantage of the present invention is that as the method does not need for a correct application, a specialized person for sampling, then the samples can be taken anywhere, and then sent to an analysis center, and thus, the sample is transferred to the analysis center instead of the patient, as in the case of endoscopies. Table 1 comparatively shows the advantages of the present invention over the solutions offered by the prior art.

TABLE 1

| Urease Test | | | |
|---|---|---|---|
| (current method | Test of Expired air | Antigens from fecal | |
| available to the | (used in some | samples (used in | Nested qPCR |
| entire population) | private centers) | private centers) | (proposed method) |
| Endoscopy is | No prior method is | No prior method is | No prior method is |
| required | required | required | required |
| Invasive | Non-invasive | Non-invasive | Non-invasive |
| Expense | Expense | Cheap | Cheap |
| Low sensibility | High sensibility | High sensibility | High sensibility |
| No detection is | No detection of a | No detection is | Detection is |
| allowed to a | genotype is allowed | allowed to a | allowed to a |
| genotype | | genotype | genotype |

### BRIEF DESCRIPTION OF THE FIGURES

FIG. 1: Illustrated assembly of original bottles, labeled and containing the fecal material.

FIGS. 2A-2C: Sample purification steps DNA protocol Kit Mag-Bind Stool DNA® (Omega).

FIGS. 3A-3D: Shows DNA integrity from 68 fecal samples from subjects in the region, visualized in 1% agarose gel.

FIG. 4: Shows the design of NCBI starters, indicating the specific parameters for the design of the UreC gene of H. pylori, through the PubMed_NCBI program. The same conditions were used for the design of the CagA gene starters.

FIG. 5: Shows the result of the amplification of the UreC gene in 5 positive fecal samples (RUT and histology). In addition, H. pylori DNA (26695) was used as control.

FIG. 6: Melt curve for the UreC gene, made with 5 positive samples for H. Pylori, in addition to the reference strain 26695. It is observed that all samples amplify at the same dissociation temperature, 78° C. (Tm).

FIG. 7: A. 2.5% agarose gel showing the amplification of the UreC gene in samples 2, 18, 21 and 43, in addition to H. pylori DNA (control+), with external splitters (left side) and internal partiers (right side). B. Amplification graph by qPCR that clearly shows how it amplifies the H. pylori genomic DNA (control+), using the external UreC starters, while the amplification of the samples evaluated is not yet observed. C. Amplification graph by qPCR, using the internal primers for UreC, showing the DNA control amplification (+) and samples 2 and 21, which were positive, while samples 18 and 43 were negative. D. Melt curve of the control DNA amplification with external splitters. E. Melt curve of positive samples and control (+), with internal splitters.

### DETAILED DESCRIPTION OF THE INVENTION

The present invention relates to a method for determining the presence of genetic material (DNA) of the bacterium Helicobacter pylori in fecal samples of subjects, by means of the nested-qPCR technique, using the UreC and CagA genes as markers, and also allowing genotyping said genes. Bacteria by determining the presence of this last gene. In particular, cagA positive (+) strains are associated with the development of gastric cancer.

For this, DNA is extracted from fecal samples, using a commercial kit designed for this type of samples, Qlamp Stool mini kit (Qjagen), or E.Z.N.A.® Stool DNA Kit (Omega Blotek)}. Subsequently, once the genetic material has been extracted, from 100 ng of DNA, it is amplified by conventional PCR, and using specific external primers (see table 2, External UreC and External CagA splitters), a region of 224 and 235 bp for the UreC and CagA genes respectively. Subsequently, 2 μl of this PCR product is used for a second round (nested) using qPCR using internal primers (see table 2, internal Urec and internal CagA splitters), which amplify a region of 127 and 100 pb respectively. In particular, the UreC gene is used only as a marker to detect infection, while the CagA gene is used as a marker to detect the presence of a more aggressive strain of the bacteria, and related to gastric cancer.

The primers (see table 2) of the present invention are small, specific DNA sequences of 18-25 nucleotides that recognize and bind to a target DNA sequence contained in the genes for which they were targeted, in this case, the genes UreC and cagA.

By the present method, in a time of approximately 3 hours, including the use of the conventional PCR technique, plus the qPCR technique (collectively, called nested qPCR), and using the primers for UreC, the subject whose fecal sample is detected is detected is analyzed, presents or not, an infection by Helicobacter pylori. In case of presenting the infection, a second test is done, which is identical to the previous one, but using the starters for cagA, and it is established whether the subject is positive or negative for the aggressive strain CagA+.

### EXAMPLE

Protocol

1: Extraction of DNA from Fecal Material.

Fecal samples were obtained from symptomatic digestive subjects who had undergone upper digestive endoscopy (DAS).

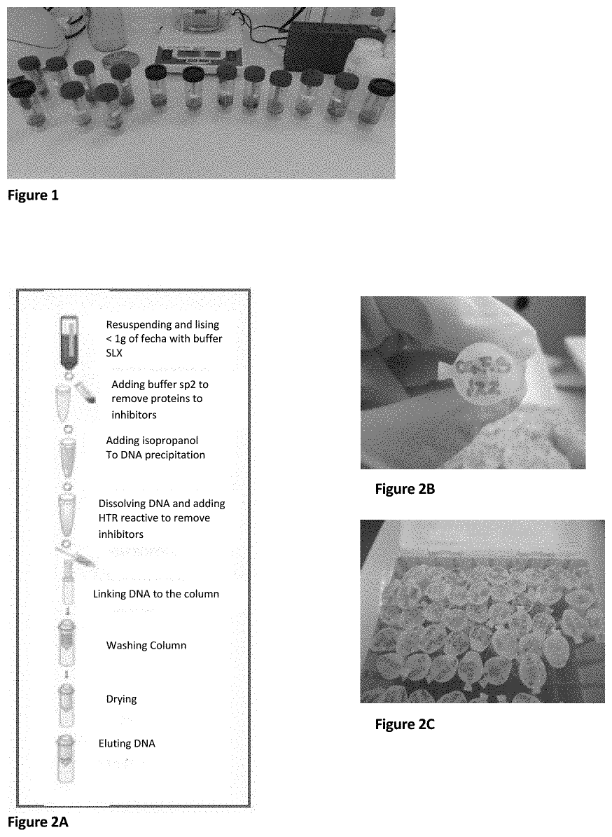

The samples were collected during a period of 3 months, and with stool samples (about 5 g), in a 30 ml wide tube, sealed, with a screw cap, made of disposable material, containing 5 ml of a nucleic acid stabilizer, preferably RNA Later®) (FIG. 1).

Each tube was properly labeled, and approximately 200-300 mg of fecal material was processed per case, with the Qlamp Stool mini kit (Qjagen) or the Mag-Bind Stool DNA® (Omega) kit, that were used according to the manufacturers' protocols and in sterile conditions. Briefly, the procedure begins with the thawing of the fecal sample and the transfer of 200-300 mg of this fecal material to an eppendorf tube, which is incubated in a lysis solution containing NaCl, EDTA and SDS; Proteins and insoluble particles are eliminated, followed by rapid washing steps to eliminate contaminating traces, finally the purified DNA is eluted with 50 μl of nuclease-free water.

These kits provide a quick and easy method of isolation of genomic DNA from fecal samples containing high humic acid content (FIG. 2).

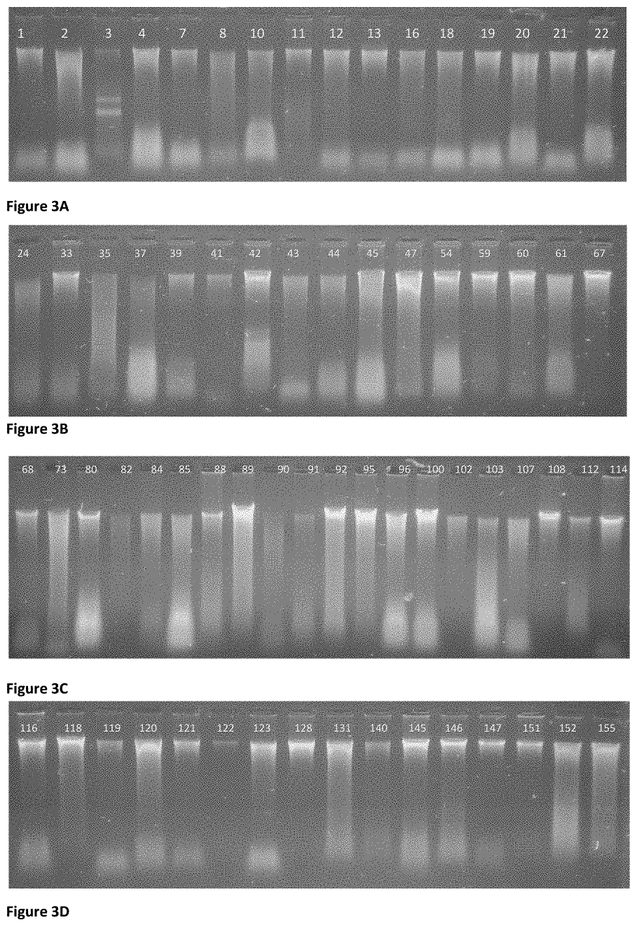

Each purified DNA was quantified, measuring the absorbance at 260 nm to determine its concentration, and at 280 nm to measure its purity, using a UV spectrophotometer (UV-9200, RAILEIGH). The integrity of the purified DNA was determined by electrophoresis in 0.8% agarose gel stained with ethidium bromide (FIG. 3).

2. Design of Partidores for Nested PCR

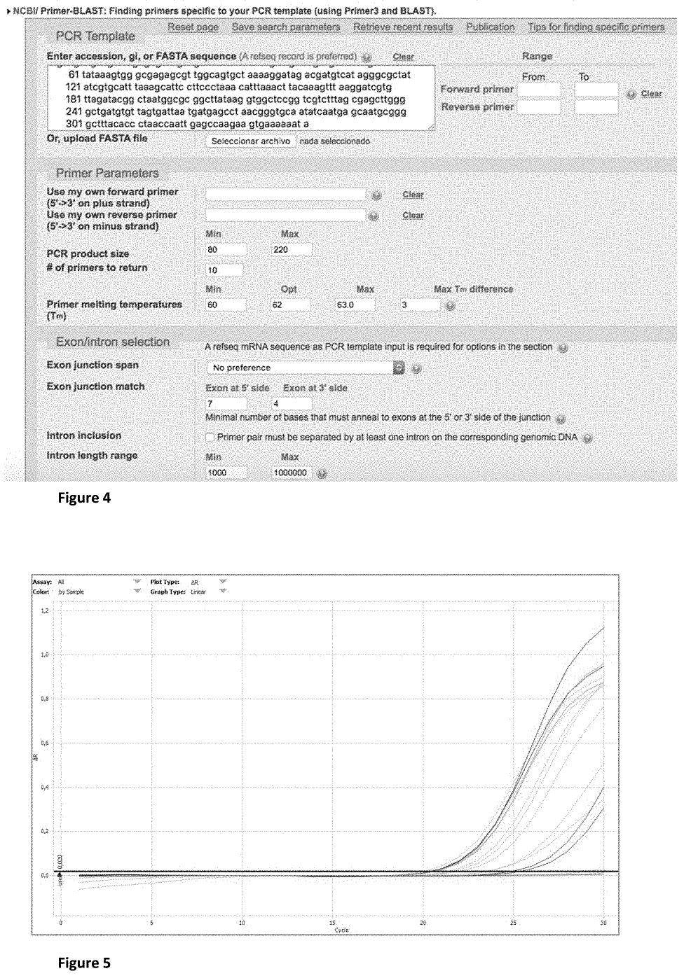

The splitters were designed in the NCBI-BLAST-PRIMERS BLAST database at http://www.ncbi. nim.n.h.gov/(FIG. 4). Particles were designed for the UreC and CagA genes, an external pair and an internal pair for each of them (see table 2).

TABLE 2
Pair of Particles for Nested PCR.

| | | Amplicon |
|---|---|---|
| Gene | Primer Sequence | size (pb) |
| External | 1) F Ex: AGCTATAAAGTGGGCGAGAG | 224 pb |
| UreC | (sequence SEQ ID No.: 1). | |
| | 2) R Ext: | |
| | ATTGCACCCGTTAGGCTCCAT | |
| | (sequence SEQ ID No.: 2). | |
| Internal | 3) F In: GCGTTGGCAGTGCTAAAAGG | 127 pb |
| UreC | (sequence SEQ ID No: 3) | |
| | 4) R In: AGCCGTATCTAACACGATCC | |
| | (sequence SEQ ID No: 4) | |
| External | 5) F Ex: 5′- | 224 pb |
| CagA | CTGGTGGGGATTGGCTTGAT-3′ | |
| | (sequence SEQ ID No.: 5). | |
| | 6) R Ext: 5′- | |
| | GCGACTCCCTCAACATCCAA-3′ | |
| | (sequence SEQ ID No.: 6). | |
| Internal | 7) F In: 5′- | 127 pb |
| UreC | AGAAACGCTCAATCAAGAGCCAA-3′ | |
| | sequence SEQ ID No.: 7) | |
| | R In: 5′- | |
| | AAGCAAATCTCTAGCTTCAGGCG-3′ | |
| | (sequence SEQ ID No: 8) | |

3. Nested-qPCR Assay in Fecal Samples

The UreC gene was amplified from the purified DNA from the fecal samples. As control, DNA of H. pylori strain 26695 (Helicobacter pylori ATCC 700392, control strain) was used.

For this, nested-qPCR was carried out consisting of the following two steps:

Conventional PCR: 2 μl of the stock of each sample of purified DNA (100 ng/μl) were incubated in the presence of 5 μl of 5× Buffer; 1.5 μl MgCb 25 mM; 0.5 μl 10 mM dNTPs; 1 μl of each splitter (see table 2) (10 μM each); 0.75 μl Taq pol, in a final volume of 25 μl. The reaction was carried out in a thermal cycler model AXYGEN MAXYGENE GRADIENT, with the following cycling conditions: denaturation at 94° C. for 5 min; followed by 25 cycles divided into denaturation 94° C., 45 sec; Hybridization at 59° C. 45 sec.; and extension at 72° C., 45 sec. Subsequently, an extension of 10 min was carried out at 72° C.

From this amplicon (PCR product) qPCR is performed.

Real-Time PCR (qPCR): The amplifications of the samples were duplicated and a positive control of H. pylori and a negative control (nuclease-free water) were placed in each assay to evaluate the quality of the assay. 2 μl of a 1/10 dilution of the above PCR product was added and incubated in the presence of 5 μl of SYBR Green kit 2× (this reagent contains: 2× Buffer, 2.5 mM MgCl 2. 0.4 mM dNTPs and 0.2 U of Taq polymerase), and 0.1 μM of each specific splitter (see table 2, internal splitters) (IOMm), in a final volume of 10 μl. Cycling conditions were as follows: initial denaturation, 95° C., 5 min, followed by 40 PCR cycles with denaturation at 95° C., 10 sec and extension at 60° C., 30 sec. The results were visualized using the Eco™ Real-Time PCR System Ilumina® program.

The results were compared with the results obtained by reference tests, RUT and Histology, in the subjects evaluated.

Data Analysis

The data were analyzed directly from the Eco Software v4.1 PCR System and with the XLSTAT Version 2.06 program, calculating sensitivity, specificity, positive and negative predictive value of the PCR test to detect infection of symptomatic digestive subjects in fecal samples. The statistical significance was established with the X² test (p=0.05).

Results

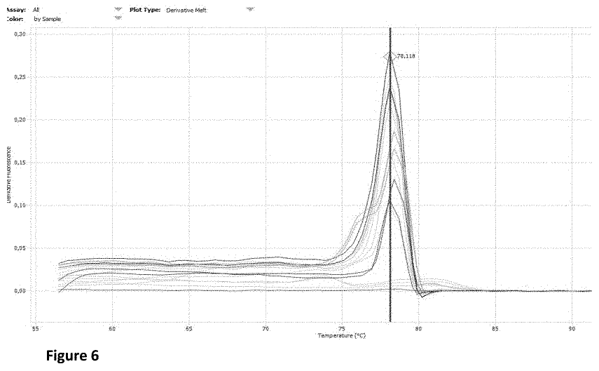

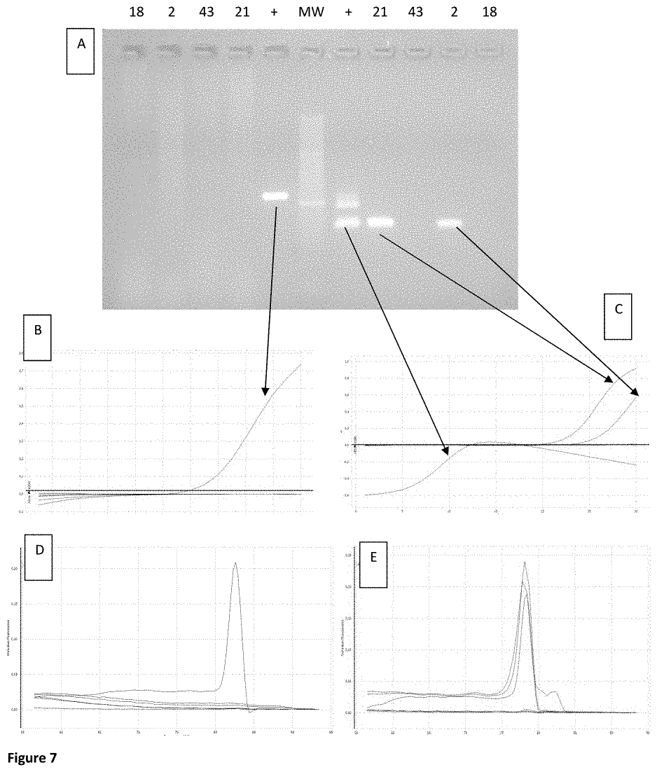

The amplification of H. pylori DNA in the fecal samples and in the control DNA, began in all samples after cycle 20 of qPCR. All the samples that showed a maximum in the “melt” curve at 78° C. were considered positive, while the absence of a maximum at that temperature, or the appearance of a curve at another temperature, was considered negative. The positive control amplified in each of the tests and the negative control did not pass the threshold line, that is, it did not have any amplification in the graphs, which validated the results (FIGS. 5 and 6).

All results were tabulated and compared with the rapid urease test (RUT) and histology (Table 3). The results of each test are shown in Tables 4, 5 and 6.

According to this, it is appreciated that the best test to detect the presence of H. pylori, is the nested-qPCR method, with a sensitivity of 100% (Table 7).

TABLE 3
60 coded fecal samples from subjects with digestive symptoms.

| | | | | qPCR | qPCR |
|---|---|---|---|---|---|
| No | Age/Genus | RUT | Histology | ureC | cagA |
| 1 | 70/M | + | + | + | + |
| 2 | 55/F | − | + | + | + |
| 3 | 68/M | − | + | + | + |
| 4 | 65/M | + | + | + | + |
| 5 | 44/F | + | + | + | + |
| 6 | 64/M | − | + | + | + |
| 7 | 70/F | − | + | + | + |
| 8 | 61/F | + | + | + | − |
| 9 | 61/F | − | + | + | + |
| 10 | 65/F | + | + | + | − |
| 11 | 81/M | − | + | + | + |
| 12 | 42/F | + | + | + | + |
| 13 | 76/M | − | − | − | ND |
| 14 | 74/M | − | + | + | − |
| 15 | 62/F | − | + | + | + |
| 16 | 39/F | + | + | + | + |
| 17 | 67/M | − | − | − | ND |
| 18 | 81/F | − | + | + | + |
| 19 | 69/F | − | − | − | ND |
| 20 | 60/F | + | + | + | + |
| 21 | 42/F | + | + | + | + |
| 22 | 64/F | −− | + | + | + |
| 23 | 65/F | − | + | + | + |
| 24 | 54/F | + | + | + | + |
| 25 | 62/F | + | + | + | + |
| 26 | 64/M | + | + | + | + |
| 27 | 40/F | + | + | + | + |
| 28 | 49/F | + | + | + | − |
| 29 | 48/F | − | − | − | ND |
| 30 | 41/M | − | + | + | − |
| 31 | 54/F | − | + | + | − |
| 32 | 43/F | + | + | + | + |
| 33 | 22/M | − | + | + | + |
| 34 | 39/F | + | + | + | + |
| 35 | 60/F | − | + | + | + |
| 36 | 32/F | + | + | + | + |
| 37 | 44/M | + | + | + | + |
| 38 | 52/F | − | − | − | − |
| 39 | 49/F | − | + | + | + |
| 40 | 74/F | + | + | + | + |
| 41 | 78/M | + | + | + | + |
| 42 | 42/F | + | + | + | + |
| 43 | 53/M | + | + | + | + |
| 44 | 29/F | + | + | + | + |
| 45 | 28/F | + | + | + | + |
| 46 | 45/F | − | + | + | − |
| 47 | 53/F | + | + | + | + |
| 48 | 31/F | + | + | + | − |
| 48 | 33/F | − | − | − | + |
| 50 | 32/M | − | + | + | + |
| 51 | 59/F | − | + | + | − |
| 52 | 40/F | + | − | + | ND |
| 53 | 47/F | + | + | + | + |
| 54 | 41/F | − | + | + | + |
| 55 | 77/F | + | + | + | + |
| 56 | 17/F | − | − | + | + |
| 57 | 86/F | + | − | + | + |
| 58 | 66/F | − | + | + | − |
| 59 | 54/F | + | + | + | − |
| 60 | 44/M | − | − | − | ND |

TABLE 4
RUT Results

| | Infected | Non infected |
|---|---|---|
| RUT+ | 31 | 1 |
| RUT− | 20 | 8 |

TABLE 5
Results of Histology-Giemsa

| | Infected | Non infected |
|---|---|---|
| Histology+ | 50 | 0 |
| Histology− | 1 | 9 |

TABLE 6
Results of qPCR tests

| | Infected | Non infected |
|---|---|---|
| qPCR+ | 51 | 2 |
| qPCR− | 0 | 7 |

TABLE 7
Comparative table of RUT, Histology and
Nested qPCR to detect H. pylori.

| Parameter | RUT | Histology | qPCR |
|---|---|---|---|
| Sensibility (%) | 60.7% | 98.0% | 100% |
| Specificity (%) | 88.8% | 100% | 85.7% |
| PPV (%) | 100% | 100% | 97.4% |
| NPV (%) | 26.9% | 90% | 100% |

PPV = Positive Predictive Value
NPV = Negative Predictive Value

1. Non-invasive method for the identification of infection by Helicobacter pylori by detecting the genes UreC (Phosphoglucosamine mutase, sequence SEQ ID No.:9) and CagA (Cytotoxin associated with gene A, sequence SEQ ID NO.:10), in samples fecal, characterized in that the method comprises: a) providing a fecal sample to detect genetic material (DNA) of the bacterium Helicobacter pylori (H. pylori), b) determine by conventional PCR technique, and then using the qPCR technique (together, called nested-qPCR), the presence of DNA of the bacterium Helicobacter pylori in said fecal sample using the UreC and CagA genes as markers, i.e., from DNA extracted from the fecal sample, the H. pylori DNA is amplified by conventional PCR with selected primers from: a primer for the sense sequence UreC gene SEQ ID No.:1 and antisense sequence SEQ ID No.:2; and a splitter for the CagA gene of sense sequence SEQ ID No.:5 and the antisense sequence SEQ ID No.:6; c) perform a second PCR (qPCR) using the following primers: a splitter for the sense sequence UreC gene SEQ ID No.:3 and antisense sequence SEQ ID No.:4; and a primer for the CagA gene of sequence SEQ ID No. 7 and the antisense sequence SEQ ID No.:8, and subsequently, confirming an H. pylori infection when determining the presence of a maximum corresponding to the fragment of DNA amplified from said bacteria.

2. Particle generator for UreC gene characterized in that it comprises the sense sequence SEQ ID No.:1 and the antisense sequence SEQ ID No.:2.

3. Particle generator for UreC gene characterized in that it comprises the sense sequence SEQ ID No.:3 and the antisense sequence SEQ ID No.:4.

4. Particle for CagA gene characterized in that it comprises the sense sequence SEQ ID No.:5 and the anti-sense sequence SEQ ID No.:6.

5. Particle for CagA gene characterized in that it comprises the sense sequence SEQ ID No.:7 and the antisense sequence SEQ ID No.:8.

6. Kit for the identification of Helicobacter pylori infection by detecting the UreC and CagA genes in fecal samples characterized in that it comprises one or more of the primers according to claims 2 to 5 in a conventional PC, and then in a qPCR.


SEQUENCE LISTING (10 sequences)

SEQ ID NO: 1 - DNA, 20 bp, artificial sequence
  (1)...(20): Primer
agctataaagtgggcgagag

SEQ ID NO: 2 - DNA, 20 bp, artificial sequence
  (1)...(20): Primer
attgcacccgttaggctcat

SEQ ID NO: 3 - DNA, 20 bp, artificial sequence
  (1)...(20): Primer
gcgttggcagtgctaaaagg

SEQ ID NO: 4 - DNA, 20 bp, artificial sequence
  (1)...(20): Primer
agccgtatctaacacgatcc

SEQ ID NO: 5 - DNA, 20 bp, artificial sequence
  (1)...(20): Primer
ctggtggggattggcttgat

SEQ ID NO: 6 - DNA, 20 bp, artificial sequence
  (1)...(20): Primer
gcgactccctcaacatccaa

SEQ ID NO: 7 - DNA, 23 bp, artificial sequence
  (1)...(23): Primer
agaaacgctcaatcaagagccaa

SEQ ID NO: 8 - DNA, 23 bp, artificial sequence
  (1)...(23): Primer
aagcaaatctctagcttcaggcg

SEQ ID NO: 9 - DNA, 341 bp, Helicobacter pylori
  (1)...(341): UreC gene
gaagaagaagaaagagcgattgaagaaatctttcacaatgaagaattgttgcattctagctataaagtgggcgagagcgttggcagtgctaaaaggatagacgatgtcatagggcgctatatcgtgcatttaaagcattccttccctaaacatttaaacttacaaagtttaaggatcgtgttagatacggctaatggcgcggcttataaggtggctccggtcgtctttagcgagcttggggctgatgtgttagtgattaatgatgagcctaacgggtgcaatatcaatgagcaatgcggggctttacaccctaaccaattgagccaagaagtgaaaaaata

SEQ ID NO: 10 - DNA, 3519 bp, Helicobacter pylori
  (1)...(3519): CagA gene
atgactaatgaaaccattgatcaaacaacaacaccagatcaaacagattttgttccgcaacgatttatcaataatcttcaagtagcttttattaaagttgataacgctgtcgcttcatttgatcccgatcaaaaaccaatcgttgataagaatgatagggataacaggcaagcttttgagaaaatctcgcaactaagggaagaatacgccaataaagcgatcaaaaatcctgccaaaaaggatcagtatttttcagactttatcaataagagcaatgatttaattaacaaagacaatctcattgctgtagattcttctgtagagagctttcggaaatttggggatcagcgttaccaaatttttacgagttgggtgtcccttcaaaaagatccgtctaaaatcaacacccaacaaatccaaaattttatggaaaatatcatacaaccccctatctctgatgataaggaaaaagcggagtttttgaggtctgccaaacaatcttttgcaggaattatcatagggaaccaaatccgatcggatgaaaaattcatgggcgtgtttgatgaatctttgaaagcaaggcaagaagcagaaaaaaatgcagagcctgctggtggggattggcttgatatttttttatcatttgtgtttaacaaaaaacaatcttccgatctcaaagaaacgctcaatcaagagccaaggcctgattttgaacaaaatttagccactaccaccaccgacatacaaggcttaccgcctgaagctagagacttgcttgatgaaaggggtaatttttttaaattcactcttggtgatgtggaaatgttggatgttgagggagtccccgacaaggatcctaattacaagttcaatcaattattgatccacaataacgctttatcttctatgctaatggggagtcatagtaacatagaacctgaaaaggtttcattattgtatggggataatggtggtcctgaagctaggcatgattggaacgctaccgttggttataaagaccaacaaggcaacaatgtggccacactcatcaatgcgcatcttaataacggcagcgggttaatcatagcgggtaatgaaaatgggattaaaaaccctagcttctacctctacaaagaagatcaactcacaggcttgaaacaagcaatgagtcaagaagagatccaaaacaaagtggatttcatggaatttcttgcaaaaaacaacgctaaattagacaacttgagcgagaaagagaaagaaaaattccaaactgagattggaaatttccaaaaagaccgtaaggcttatttagacgcactagggaatgatcacattgcttttgtttctaaaaaagacccaaaacatttggctttggttactgagtttggtaatagggaagtgagctatactctcaaagattatgggaaaaaacaagataaagctttagatggggagacaaaaaccactcttcaaggtagcctaaaatatgatggcgtgatgtttgtcaattattccaatttcaaatacaccaacgcctccaagagtcctgataagggtgtaggcactacgaatggcgtttcccatttggaagcaaattttagtaaggtagctgtctttaatttgcctaatttaaataatctcgctatcactaattatataaggcgggatttagaagataaattgtgggctaaaggattgtccccacaagaagctaataagctcatcaaagactttttgaacagcaacaaagaattgcttggaaaagtttcaaacttcaataaagctgtagctgaagctaaaaacacaggcaattatgacgaagtgaaaaaagctcagaaagatcttgaaaaatctctaaggaaacgagagcatttagagaaagaagtagcgaaaaaattggagagcagaaacgacaacaaaaatagaatggaagcaaaagctcaagctaacagccaaaaagataagatttttgcacttatcaatcaagaggctagtaaggaagcaagagcggccgctttcgatccgaatcttaaaggcatcaggagcgaattgtctgataaacttgaaaatatcaacaagaatttgaaagactttggcaaatcttttgatgaactcaaaagtggcaaaaataatgatttcagcaaggcagaagaaacgctaaaagcccttaaagactcggtgaaagatttaggcatcaatccagaatggatttcaaaaattgaaaaccttaatgcagctttgaatgatttcaaaaatggcaaaaataaggattccagcaaggtaacacaagcaaaaagcgaccttgaaaattccattaaggatgtgatcattaatcaaaagataacggataaagttgacaatctcaatcaggctgtatcagagactaaattaacaggcgatttcagtaaggtagagcaagccctagccgaactcaaaagcttgtcattggatcttggaaaaaattctgatctacaaaaatccgttaaaaatggtataaatggaaccctagtcggtaatgggttgtctaaaacagaagccacaacgctcaccaaaaatttttcggacatcaggaaagaattgaacgagaaattatttggaaattccaataacaataataatggactcaaaaacaacacagagcctatttatgctcaagttaataaaaagaaagcaggacaagcaactagccctgaagagcccatttacgctcaagttgctaaaaaggtgagttcaaaaattgaccaactcaacgaagctacatcggcaataaatagaaaaattgaccggattaacaaaattgcatcagcaggtaaaggagtgggcggtttcaatggagcagggcgatcagctagccctgaacccatttacgctacaattgattttgatgaggcaaatcaagcaggcttccctttgagaagaagtgctgcagttaatgatctcagtaaagtagggctttcaagggaacaagaattgactcgtagaattggcgatctcagtcaggcagtgtcagaagctaaaacaggtcattttgacaacctagaacaaaagatagatgaactcaaagattctacaaaaaagaatgctttgaagctatgggttgaaagcgcaaaacaagtgcctactggtttgcaagcgaaattggacaattacgctactaacagccacacacgcattaatagcaatgtccaaagtggaacaatcaatgaaaaagcgaccggcatgctaacgcaaaaaaaccctgagtggctcaagctcgtgaatgataagatagttgcacataatgtgggaagcgctcatttgtcagagtatgataaaattggattcaaccaaaagaatatgaaagattattctgattcgttcaagttttccaccaagttgaacaatgccgtaaaagacattaagtctagctttgtgcaatttttaaccaacacattttctacaggatcttacagcttgatgaaagcaaatgtggaacatggagtcgaaaacactacaaaaagtggtttccagaaatcttaa